(19) United States
(12) Patent Application Publication
(10) Pub. No.: US 2015/0087964 A1
(43) Pub. Date: Mar. 26, 2015

(54) METHOD FOR OPTIMIZING A MEDICAL IMAGING ACQUISITION

(71) Applicant: SIEMENS AKTIENGESELLSCHAFT, Muenchen (DE)

(72) Inventor: Miriam Keil, Erlangen (DE)

(73) Assignee: SIEMENS AKTIENGESELLSCHAFT, Muenchen (DE)

(21) Appl. No.: 14/493,624

(22) Filed: Sep. 23, 2014

(30) Foreign Application Priority Data
Sep. 23, 2013 (DE) 102013219042.5

(51) Int. Cl. G01R 33/567; G01R 33/56; G01R 33/54; A61B 5/055; G01R 33/58

(52) U.S. Cl. CPC G01R 33/5673; A61B 5/055; G01R 33/58; G01R 33/543; G01R 33/5608
USPC 600/413

(57) ABSTRACT

In a method for the optimization of an imaging acquisition of an object by a medical imaging apparatus, a first time period is determined, within which a calibration process of the medical imaging apparatus is executed, a second time period is determined, within which a breathing process for at least one breathing command of a recording protocol is executed, wherein the breathing process is executed at least in part at the same time as the calibration process, and a point in time for an initiation of the imaging acquisition is determined, such that at this point in time, the calibration process and the breathing process have been completed.

### BACKGROUND OF THE INVENTION

1. Field of the Invention

The present invention relates to a method for optimizing an imaging acquisition of an object under examination by operation of a medical imaging apparatus, as well as a corresponding optimization unit and a medical optimization system and a non-transitory data storage medium encoded with programming instructions, that enable the execution of a method of this type.

2. Description of the Prior Art

Optimization of imaging acquisitions, in particular a temporal optimization of data acquisitions of this type, is a widespread field of activity in clinical medical applications.

In the daily operations of a medical clinic, an efficient and targeted optimization of imaging acquisitions can present an extremely complex problem, particularly if multiple boundary conditions are to be taken into account.

### SUMMARY OF THE INVENTION

An object of the invention is to provide a method that facilitates optimization of a medical imaging acquisition, and with which a determination of the time required for the image data acquisition, dependent on various boundary conditions, is possible in a simple manner.

The above object is achieved in accordance with the invention by a method for optimizing an imaging acquisition of an object under examination by operation of a medical imaging apparatus, that includes the following steps.

A first time period is determined in the control computer of the imaging apparatus, within which a calibration process is executed for at least one adjustment of the medical imaging apparatus.

A second time period is determined in the control computer, within which a breathing process is executed for at least one breathing command of an image data acquisition protocol, wherein the breathing process is executed at least in part at the same time as the calibration process.

A point in time for an initiation of the imaging acquisition is determined in the control computer, such that the calibration process and the breathing process both have been completed at this point in time.

A medical imaging apparatus is a device, preferably an electronic and/or data technology device, for acquiring, processing, evaluating and/or storing image information in the form of image data. In order to acquire the image data, acoustic methods such as ultrasound (US), emission methods such as emission computed tomography (ECT) and positron emission tomography (PET), optical methods, radiological methods such as X-ray tomography and computed tomography (CT), are used, but the acquisition can also occur by magnetic resonance tomography (MR or MRT), or by combined methods. The medical imaging apparatus can deliver 2-dimensional (2D) or multi-dimensional, such as 3-dimensional (3D) or 4-dimensional (4D) image data, which are stored and/or processed in various formats. The medical imaging apparatus can be used in a diagnosis, for example in a medical diagnosis.

First, a first time period is automatically determined, within which a calibration process for at least one adjustment of the medical imaging apparatus is executed. As used herein, such an adjustment means an adjustment of at least one component of the imaging apparatus prior to the imaging acquisition. For example, for a magnetic resonance acquisition using a magnetic resonance apparatus, the calibration process may include a receiver calibration, a frequency calibration and/or a transmitter calibration. The receiver calibration includes, e.g. an adjustment of a receiver dynamic for an analog-digital converter; the frequency calibration includes, e.g. an adjustment of the frequency of a radio frequency system to the resonance frequency of the nuclear spins for the generation of magnetic resonance signals; and the transmitter calibration includes, e.g. an adjustment of a transmission power for the radio frequency pulses. During the at least one adjustment of the calibration process, parameters can also be determined that are dependent on the object under examination.

The first time period, within which the calibration process is executed, is either predetermined, or can be estimated in accordance with the invention. However determined, the first time period must ensure that after completion of the first time period, the calibration process is also completed.

A breathing process for at least one breathing command of an image data acquisition protocol is executed within the second time period that is determined. Breathing commands of this type are used in many imaging acquisitions, in which movements in an interior of the object under examination, such as the motion of a patient's heart, cause blurring in the image that is reconstructed from the imaging data acquisition. Breathing commands normally include time periods in which the patient inhales and/or time periods in which the patient holds his or her breath, and/or time periods in which the patient exhales. The breathing process thus includes at least one breathing command that is communicated to the patient. Preferably, the respiratory state of the patient achieved as a result of the breathing command should be maintained during the imaging acquisition.

With automatic breathing commands, the second time period is normally known, because the breathing commands comply with a previously established acquisition protocol. If this second time period is not known, then it can be measured in advance.

According to the invention, the breathing process occurs at least in part at the same time as the calibration process. It is thereby ensured that it is not first necessary to wait until the calibration process is finished, before the breathing process can be started, or vice versa.

The point in time for starting the imaging acquisition is then determined such that at this point in time, the calibration process and the breathing process have been completed.

The invention makes use of the at least partial simultaneity (overlap) of the calibration process and the breathing process for a temporal optimization of the initiation of the imaging acquisition. This results in shorter waiting periods, an increased patient turnover, and a more efficient workflow.

In an embodiment, a completion point for the first time period corresponds to a completion point for the second time period. The point in time for the initiation of the imaging acquisition thus is determined such that at this point, the calibration process and the breathing process are completed at the same time. This results in a further temporal optimization for the initiation of the imaging acquisition, and thus to a shortening of the actual recording time as well.

In a further embodiment, at least one of a time period between the completion point for the first time period and the initiation of the imaging acquisition, and a time period between the completion point for the second time period and the initiation of the imaging acquisition, is minimized. The waiting period between the completion of the calibration process and/or the completion of the breathing process, and the initiation of the actual creation of an image is thus further reduced. In an embodiment, the imaging acquisition is initiated immediately after the completion point for the first time period and/or the completion point for the second time period.

In a preferred embodiment, with execution of at least two adjustments, the adjustments are executed in a sequence, such that adjustments with a higher noise level are carried out initially, and adjustments with a lower noise level are carried out at the end of the calibration process. At least one first adjustment thus is allocated to a first noise level, and a second adjustment is allocated to a second noise level, with the first noise level being higher than the second noise level. Such a sorting of the adjustments according to a noise volume within the calibration process ensures, in combination with the at least partial simultaneity of the calibration process and the breathing process, that background noise, resulting from the calibration process, have as little acoustic affect as possible on the breathing commands for the breathing process.

The present invention also encompasses an optimization unit for optimizing an imaging acquisition of an object under examination by means of a medical imaging apparatus is also provided.

The optimization unit includes a processor that is designed to execute the following steps: determination of a first time period, within which a calibration process is executed by the processor, for at least one adjustment of the medical imaging apparatus; determination of a second time period, within which a breathing process for at least one breathing command of an image data acquisition protocol is executed by the processor; wherein the breathing process is executed at least in part at the same time as the calibration process; and determination of a point in time for an initiation of the imaging acquisition by the processor, such that the calibration process and the breathing process have been completed at this point in time.

The invention also encompasses a medical optimization apparatus that includes such an optimization unit and at least one medical image data acquisition unit.

The present invention also encompasses a non-transitory, computer-readable data storage medium encoded with programming instructions that, when the storage medium is loaded into a computerized processor of an image acquisition unit, causes the processor to function as an optimization unit as described above, in order to implement one or more of the above-described embodiments of the method according to the invention. The programming instructions may require additional items, such as libraries or other auxiliary functions, in order to cause the embodiments of the method to be implemented.

The electronically readable storage medium may be a DVD, a magnetic tape, or a USB stick, on which electronically readable control information, in particular software, is stored.

Advantages and embodiments of the optimization unit according to the invention, the medical optimization apparatus according to the invention, and the electronically readable storage medium according to the invention correspond substantially to the advantages and embodiments of the method according to the invention, as described above.

### BRIEF DESCRIPTION OF THE DRAWINGS

FIG. 1 schematically illustrates a medical optimization apparatus according to the invention.

FIG. 2 is a flowchart of an embodiment of the method according to the invention.

FIG. 3 shows an example of an optimization of an imaging acquisition in accordance with the invention.

### DESCRIPTION OF THE PREFERRED EMBODIMENTS

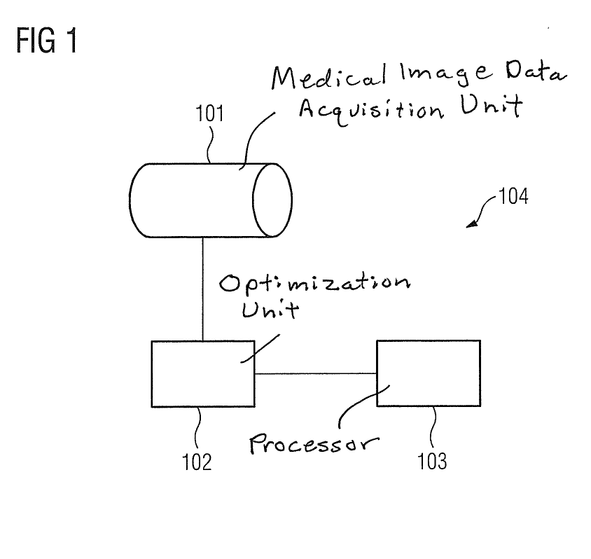

FIG. 1 shows a medical optimization apparatus 104 according to the invention. The medical optimization system 104 has an optimization unit 102 and a medical image data acquisition unit 101, is equipped for operation in order to acquire medical image data from an object under examination.

The optimization unit 102 further has a processor 103. The processor 103 itself can be embodied in the medical image data acquisition unit 101.

The medical image data acquisition unit 101 is designed here as a magnetic resonance scanner. Alternatively, the medical image data acquisition unit 101 can be a combined magnetic resonance-positron emission tomography apparatus, or other medical imaging apparatuses 101 that appear appropriate to those skilled in the art.

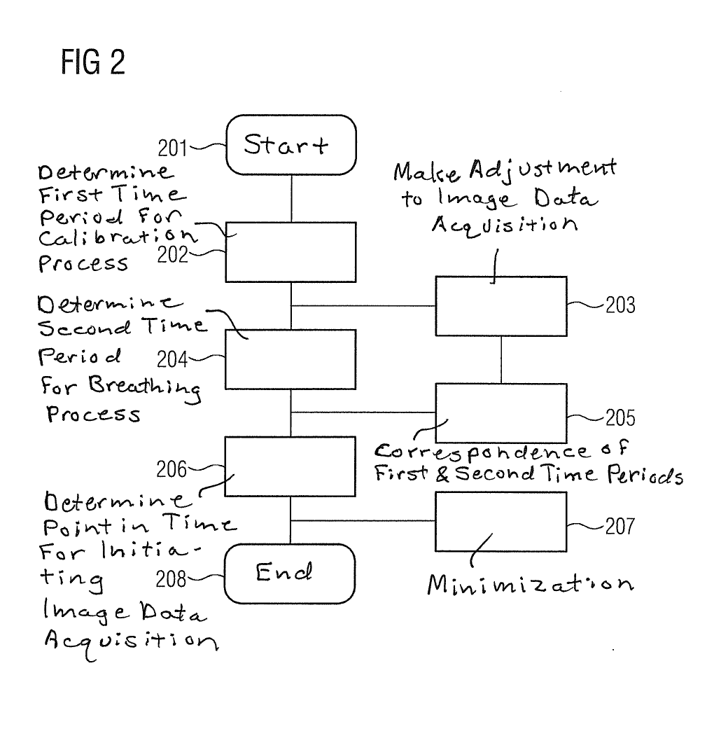

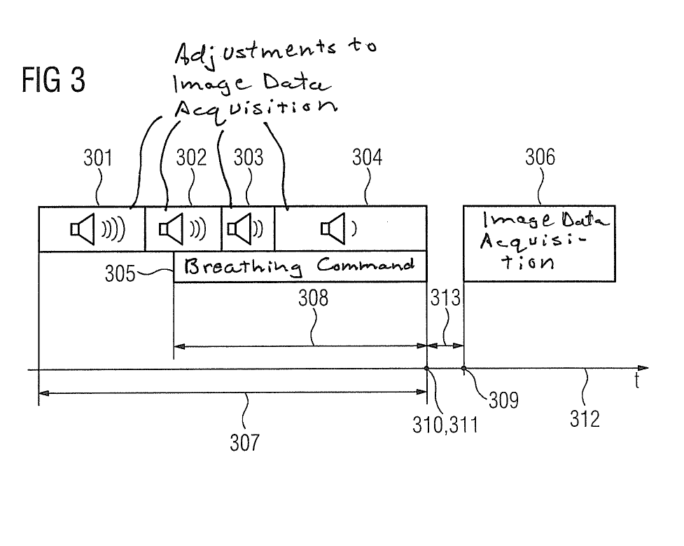

FIG. 2 shows a flowchart for an embodiment of the method according to the invention. The method includes the steps 201-208. In the following description of the steps 201-208, components are referenced as we show in FIGS. 1 and 3.

The steps 201-208 are executed by the evaluation unit 103 of the medical optimization system 104.

A first step 201 indicates the start of an optimization of an imaging acquisition 306 of an object under examination using the medical image data acquisition unit 101.

A determination of a first time period 307, within which the one calibration process for at least one adjustment 301, 302, 303, 304 of the medical image data acquisition unit 101 is executed, occurs in step 202. Such an adjustment means an adjustment of at least one component prior to the imaging acquisition 306. In the example of a magnetic resonance acquisition with a magnetic resonance apparatus, the calibration process can involve a receiver calibration, a frequency calibration and/or a transmitter calibration. The first time period 307, within which this calibration process is executed, is either known, because it is automatically provided, for example, or can be estimated according to the invention, because it is manually determined, for example. It is thus ensured that after the first time period 307 is finished, the calibration process is also complete.

With an execution of at least two adjustments 301, 302, 303, 304 in step 203, the adjustments 301, 302, 303, 304 are executed in a sequence such that adjustments 301, 302, 303, 304 having a higher noise level are executed at the start, and adjustments 301, 302, 303, 304 having a lower noise level are executed at the end of the calibration process. The adjustments 301, 302, 303, 304 are thus sorted according to the level of noise produced thereby within the calibration process. This step is optional and need not necessarily be executed.

A determination of a second time period 308, within which a breathing process for at least one breathing command 305 of a recording protocol is executed, occurs in step 204, wherein the breathing process is executed at least in part at the same time as the calibration process. Breathing commands 305 of this type are used with many imaging acquisitions 306, in which movements, such as a cardiac motion of a patient, for example, in an interior of the object under examination, cause a blurring in an image of the imaging acquisition 306. With automatic breathing commands 305, the second time period 308 is normally known, because the breathing commands 305 comply with a previously defined recording protocol. If this second time period 308 is not known, then it can be measured in advance.

In step 205 the completion point 310 of the first time period 307 corresponds to a completion point 311 of the second time period 308. As a result, the point in time 309 for the initiation of the imaging acquisition can be determined such that at this point in time 309, the calibration process and the breathing process are completed at the same time. This step is optional, and must not necessarily be executed.

Step 206 includes the determination of a point in time 309 for an initiation of the imaging acquisition 306, such that at this point in time 309, the calibration process and the breathing process have been completed. The at least partial simultaneity of the calibration process and the breathing process leads to a temporal optimization of the initiation 309 of the imaging acquisition 306.

A minimization of a time period 313 between the completion point 310 of the first time period 307 and the initiation of the imaging acquisition 306, and/or a time period 313 between the completion point 311 of the second time period 308 and the initiation of the imaging acquisition 306, occurs during a step 207. By this means, the waiting time between the end of the calibration process and/or the end of the breathing process and the initiation of the actual creation of an image, thus the point in time 309 for the initiation of the imaging acquisition 306, is further reduced. Preferably, the imaging acquisition 306 is initiated immediately after the completion point 310 of the first time period 307 and/or the completion point 311 of the second time period 308, and thus, the completion point 310 of the first time period 307 and the completion point 311 of the second time period correspond to the initiation of the imaging acquisition 306.

A last step 214 indicates the completion of the optimization of an imaging acquisition 306 of an object under examination by the medical image data acquisition unit 101.

FIG. 3 shows an example of an optimization of an imaging acquisition 306. First, a first time period 307, within which a calibration process for four adjustments 301, 302, 303, 304 of the medical image data acquisition unit 101 is executed, is determined in step 202. The reference symbol 312 represents a time axis. The adjustments 301, 302, 303, 304 of the calibration process comprise, in this case for a magnetic resonance acquisition, a receiver calibration, a frequency calibration, or a transmitter calibration. The receiver calibration comprises, e.g. an adjustment of a receiver dynamic for an analog-digital converter, the frequency calibration comprises, e.g. an adjustment of a frequency for a radio frequency system to a resonance frequency of a magnetic dipole field of the magnetic resonance apparatus, and the transmitter calibration comprises, e.g. an adjustment of a transmission power for radio frequency pulses. During the at least one adjustment 301, 302, 303, 304 of the calibration process, parameters can also be determined, which are dependent on the object under examination.

In addition, the four adjustments 301, 302, 303, 304 are sorted according to a noise volume within the calibration process, as is the case in step 203. Adjustment 301 represents the adjustment with the highest noise level, and adjustment 304 represents the adjustment with the lowest noise level. Background noises resulting from the calibration process thus affect the breathing command 305 as little as possible.

Analogously to step 204, a determination of a second time period 308 takes place, within which a breathing process for at least one breathing command 305 of an image data acquisition protocol is executed, wherein the breathing process is executed at least in part at the same time as the calibration process. Breathing commands 305 normally include time periods in which the patient inhales, and/or time periods in which the patient does not breath, and/or time periods in which the patient exhales; the breathing process comprises at least one breathing command 305. Preferably, the respiratory state of the patient obtained with the breathing command 305 should be maintained during the imaging acquisition 306. With automatic breathing commands 305, the second time period 308 is normally known, because the breathing command 305 complies with a previously established acquisition protocol. If this second time period 308 is not known, it can be measured in advance.

A point in time 309 for an initiation of the imaging acquisition 306 is then determined in step 206, such that at this point in time 309, the calibration process and the breathing process have been completed. In the illustrated example 307, a completion point 310 of the first time period 307, a completion point 311 of the second time period 308, and a time period 313 between these completion points 307, 308 and the initiation of the imaging acquisition 306 are minimized.

In an embodiment, a time period between the completion point of the first time period and the initiation of the imaging acquisition, and/or a time period between the completion point of the second time period and the initiation of the imaging acquisition, is minimized, and with an execution of at least two adjustments, the adjustments are executed in a sequence, such that adjustments with a higher noise level are executed at the start of the calibration process, and adjustments with a lower noise level are executed at the end of the calibration process.

Although modifications and changes may be suggested by those skilled in the art, it is the intention of the inventor to embody within the patent warranted hereon all changes and modifications as reasonably and properly come within the scope of her contribution to the art.

I claim as my invention:

1. A method for optimizing acquisition of medical image data from a subject by operation of a medical imaging apparatus, comprising:
in a processor, determining a first time period within which a calibration process for at least one adjustment of said medical imaging apparatus is executed;
in said processor, determining a second time period, within which a breathing process for at least one breathing command of an acquisition protocol for acquiring said medical image data is executed, said breathing process being executed at least partially overlapping in time with said calibration process;
in said processor, determining a point in time for initiation of said acquisition of medical image data, at which point in time the calibration process and the breathing process have both been completed; and
from said processor, emitting an electronic signal to said medical imaging apparatus to initiate the acquisition of said medical image data.

2. A method as claimed in claim 1 comprising, in said processor, determining a completion point of said first time period to correspond to a completion point of said second time period.

3. A method as claimed in claim 1 comprising, in said processor, minimizing at least one of a time period between a completion point of said first time period and initiation of said acquisition of medical image data, and a time period between said completion point of said second time period and initiation of said acquisition of said medical image data.

4. A method as claimed in claim 1 comprising, in said calibration process, executing at least two adjustments of said medical imaging apparatus, and executing said at least two adjustments in a sequence in which an adjustment, among said at least two adjustments, having a higher noise level is executed at a start of said calibration process, and an adjustment, among said at least two adjustments, having a lower noise level is executed at an end of said calibration process.

5. An optimization unit for optimizing acquisition of medical image data from a subject by operation of a medical imaging apparatus, comprising:
a processor configured to determine a first time period within which a calibration process for at least one adjustment of said medical imaging apparatus is executed;
said processor being configured to determine a second time period, within which a breathing process for at least one breathing command of an acquisition protocol for acquiring said medical image data is executed, said breathing process being executed at least partially overlapping in time with said calibration process;
said processor being configured to determine a point in time for initiation of said acquisition of medical image data, at which point in time the calibration process and the breathing process have both been completed; and
said processor being configured to emit an electronic signal to said medical imaging apparatus to initiate the acquisition of said medical image data.

6. A medical optimization system, comprising:
a medical imaging apparatus;
a processor configured to determine a first time period within which a calibration process for at least one adjustment of said medical imaging apparatus is executed;
said processor being configured to determine a second time period, within which a breathing process for at least one breathing command of an acquisition protocol for acquiring said medical image data is executed, said breathing process being executed at least partially overlapping in time with said calibration process;
said processor being configured to determine a point in time for initiation of said acquisition of medical image data, at which point in time the calibration process and the breathing process have both been completed; and
said processor being configured to emit an electronic signal to said medical imaging apparatus to initiate the acquisition of said medical image data.

7. A non-transitory, computer-readable data storage medium encoded with programming instructions, said storage medium being loaded into a computerized processor of an image acquisition system, comprising a medical image data acquisition unit, and said programming instructions causing said computerized processor to:
determine a first time period within which a calibration process for at least one adjustment of said medical imaging apparatus is executed;
determine a second time period, within which a breathing process for at least one breathing command of an acquisition protocol for acquiring said medical image data is executed, said breathing process being executed at least partially overlapping in time with said calibration process;
determine a point in time for initiation of said acquisition of medical image data, at which point in time the calibration process and the breathing process have both been completed; and
emit an electronic signal to said medical imaging apparatus to initiate the acquisition of said medical image data.
